(19) United States
(12) Patent Application Publication
(10) Pub. No.: US 2024/0126949 A1
(43) Pub. Date: Apr. 18, 2024

(54) SHEARING PROCESS SIMULATION METHOD

(71) Applicant: INDUSTRY-ACADEMIC COOPERATION FOUNDATION GYEONGSANG NATIONAL UNIVERSITY, Gyeongsangnam-do (KR)

(72) Inventor: Man Soo JOUN, Gyeongsangnam-do (KR)

(21) Appl. No.: 18/276,939

(22) PCT Filed: Mar. 24, 2022
(86) PCT No.: PCT/KR2022/004134

§ 371 Date: Aug. 11, 2023

(30) Foreign Application Priority Data
Mar. 25, 2021 (KR) 10-2021-0039026

(51) Int. Cl. G06F 30/23; G06T 17/20

(52) U.S. Cl. CPC G06F 30/23; G06T 17/205; G06F 2111/10

(57) ABSTRACT

A shearing process simulation method may include a first step in which a finite element and a node are generated in a raw material, a second step in which a fracture surface is calculated for a sheared material with a shearing force applied to the raw material, a third step in which an element of the sheared material is divided into a first group and a second group with the fracture surface as a boundary, a fourth step in which an average value is obtained by averaging information of a second group element or a second group node included in the second group, and a fifth step in which a final fracture surface is generated by reflecting the average value in the fracture surface.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2022/004134, filed Mar. 24, 2022, which claims priority to the benefit of Korean Patent Application No. 10-2021-0039026 filed in the Korean Intellectual Property Office on Mar. 25, 2021, the entire contents of which are incorporated herein by reference.

### BACKGROUND

### 1. Technical Field

The present disclosure relates to a specific metal forming simulation technology for obtaining the finite element predictions of shearing, piercing, blanking, and the like.

### BACKGROUND ART

Traditionally, shearing, piercing, and blanking have been studied using an experimental method or a method in which experimental and theoretical approaches are combined. During the past 30 years, many numerical studies have been carried out on shearing and material fracture to elucidate the basic mechanism of conventional shearing, piercing and blanking. However, since a predicted sheared material, i.e., a numerical preform or billet cannot be reliably used for an engineering purpose in a consecutive process, the impact of shearing on a final product could not be analyzed.

### SUMMARY

The present disclosure is provided to predict results of various processes such as shearing, piercing, blanking, upsetting, etc., and in an simulation process of an automatic multi-stage metal forming process, it is important to predict the usability of a sheared material (finite element mesh system) numerically divided by shearing, piercing, etc. for the engineering analysis of the consecutive stages using an element strength degradation algorithm, a sheared surface quality control system, and the like.

The present disclosure relates to a shearing process simulation method of obtaining a numerical preform or billet that may be used for engineering analysis of a consecutive process immediately after simulating a shearing process, and may include:
a first step in which a material may be divided into a finite element mesh system composed of a set of finite elements with nodes;
a second step in which during shearing simulation, an element strength degrading algorithm and a shearing point prediction function of ductile fracture theory may be used to determine a shearing point, or a shearing boundary area. In this step, an element strength degraded area in a raw material may be generated so that the entire forming load is sharply degraded;
a third step in which a deleting target area may be determined based on the shearing boundary, and a free node that is a deleting target included in the deleting target area may be generated;
a fourth step in which a constant tensile force may be applied to all line segments connected to each other by the free node, and an unbalance force or a resistance force of the material may be reduced by placing the free node at the appropriate position or a node smoothing method of properly placing the free node using a nodal averaging scheme is applied, the free node may be located in a shearing boundary, and a mesh containing a degenerate finite element may be generated;
a fifth step in which a state variable value may be assigned to the free node as a nodal value of a non-deletion node close to each free node, and the state variable value may be assigned to the finite element defined by the free node; and
a sixth step in which a numerical preform or billet sheared in the shearing process may be formed, and a re-meshing may be performed for a mesh containing the degenerate finite element of the numerical preform or billet and thus a strength degraded finite element collected in the shearing boundary may be deleted, and a flawless mesh may be generated in terms of finite element analysis.

As another embodiment of the present disclosure, the present disclosure relates to a shearing process simulation method, and the shearing process simulation method may include: a first step in which a finite element and a node may be generated in a raw material; a second step in which a fracture surface may be calculated for a sheared material; a third step in which an element of the material may be divided into a first group and a second group with the fracture surface as a boundary; a fourth step in which an average value may be obtained by averaging information of a second group element or a second group node included in the second group; and a fifth step in which a final fracture surface may be generated by reflecting the average value of state variables in the fracture surface, wherein the final fracture surface may be recognized as a boundary surface.

A first group element or a first group node of the first group may be regenerated with the final fracture surface as the boundary surface.

A node located in the fracture surface may be recognized as a fracture surface node and is distinguished from other nodes.

A node located in the fracture surface may be recognized as a fracture surface node and the fracture surface node and a node included in the first group may be determined as remaining nodes.

Information of the second group element or the second group node included in the second group may be reflected in the final fracture surface.

A first average value may be generated by averaging information of the second group element or the second group node, a second average value may be generated by averaging fracture surface information of a node located in the fracture surface and the first average value, an Nth average value may be generated by averaging the fracture surface information and an N-1th average value, information of the final fracture surface may be generated based on the Nth average value, and the number N may be a natural number greater than or equal to 3.

The average value may be calculated in a node averaging method in which an equal tensile force may be applied to all edge defined by the second group node.

Before the final fracture surface is generated, the second group element or the second group node may not be deleted.

The second group node may be moved to a point close to the fracture surface, an imaginary mesh may be regenerated, and a nodal value of the second group node in the imaginary mesh may be replaced by a nodal value of a node closest thereto, and an elemental value of the second group element may be replaced by an arithmetic mean of the nodal value.

The present disclosure proposes a new general finite element method (FEM) to predict shearing, blanking, piercing, or the like, and when simulating a multi-stage metal forming process, the engineering analysis usability of a target numerically-sheared material can be improved.

The present disclosure can be used as a sheared surface quality control system to predict the sheared surface quality of a sheared material.

The present disclosure can overcome a numerical problem that may occur after severely damaged elements or divided edges are deleted. Furthermore, the present disclosure can be used to simulate a 3-D round-bar shearing process for generation of a numerical preform or billet.

The present disclosure may include a first step in which a finite element and a node are generated by a mesh when a material is sheared, a second step in which the finite element of the shearing band generated during shearing is processed, and a third step in which the finite element is re-meshed after shearing is completed.

A fracture surface is generated by the shearing band, a finite element and a node (hereinbelow, which will be called as a free node) at one side based on the fracture surface is determined as the deleting target area, and the free node is smoothed by the node averaging method, the tensile force assignment method, etc., and the modified or smoothed mesh may be an imaginary mesh (the mesh may be a numerical preform or billet that may not be used for analysis), and a free node to be deleted during shearing simulation may be moved to a point close to a non-free node (which finally remains in a numerical preform or billet) by the smoothing method.

The flow stress of the material may be provided as a function of effective strain, effective strain rate, temperature, and degree of damage as independent variables, and may depend on an element strength degradation function reflecting the impact of cumulative damage with respect to flow stress.

The degree of damage may be provided by modified Freudenthal damage model or a theoretical damage model, during calculation of flow stress

### BRIEF DESCRIPTION OF THE DRAWINGS

FIGS. 1A to 1E are diagrams showing the entire process of the present disclosure.

FIGS. 2 to 6 are diagrams showing another embodiment of the present disclosure.

FIGS. 7A and 7B are diagrams showing a metal forming process of a shearing band of the present disclosure.

FIGS. 8A and 8B are views showing comparison between an experimental result and a prediction result with respect to a numerical preform or billet of the present disclosure.

FIG. 9 is a view showing comparison between an experimental result and a prediction result in a geometrical aspect with respect to a symmetric surface of a numerical preform or billet of the present disclosure.

### DETAILED DESCRIPTION

The present disclosure provides a new general FEM for predicting shearing, blanking, or piercing, and the method may be important in usability of a target numerically-sheared material in automatic multi-stage metal forming. The present disclosure may be based on an element strength degradation model with a sheared surface quality control system. The element strength degradation model may ensure generality of a method, and the sheared surface quality control system may accelerate usability for a finite element analysis purpose of a sheared or blanked material. The present disclosure may be interchanged with the theory of ductile fracture and material separation may be verified experimentally. The combined sheared surface quality control system and element strength degradation model may be applied to a specific instant when the material separation occurs. In the sheared surface quality control system, a free node that should disappear after shearing or piercing may be moved to a position closest to a fractured surface for suitability to a finite element mesh. A shearing simulation may be finished by re-meshing after a free node algorithm is applied for sheared surface quality control. The present disclosure that is different from the existing method may overcome a numerical problem that may occur after a severely-damaged element or divided edge is deleted. Furthermore, the present disclosure may be used to simulate a 3D round-bar shearing process for generating a short billet. When comparing simulated results and experimental results, the present disclosure can simulate a metal forming process with respect to a material finely sheared or blanked in a compressive stress state, easily, systematically and accurately.

Most metal forming begins with a shearing process that may include piercing or blanking, so that metrical analysis of shearing, piercing, or blanking may be important. Strain hardening by shearing and deformation of an initial material may have a non-negligible impact on the consecutive stages. A sheared material, i.e., a numerical preform or billet, may be evaluated in terms of suitability for a finite element analysis in the consecutive engineering step on the basis of macroscopic characteristics of a predicted sheared surface. Therefore, the sheared surface quality, including rollover, inclined angle, earing, etc., can be predicted in terms of the usability and precision forming,

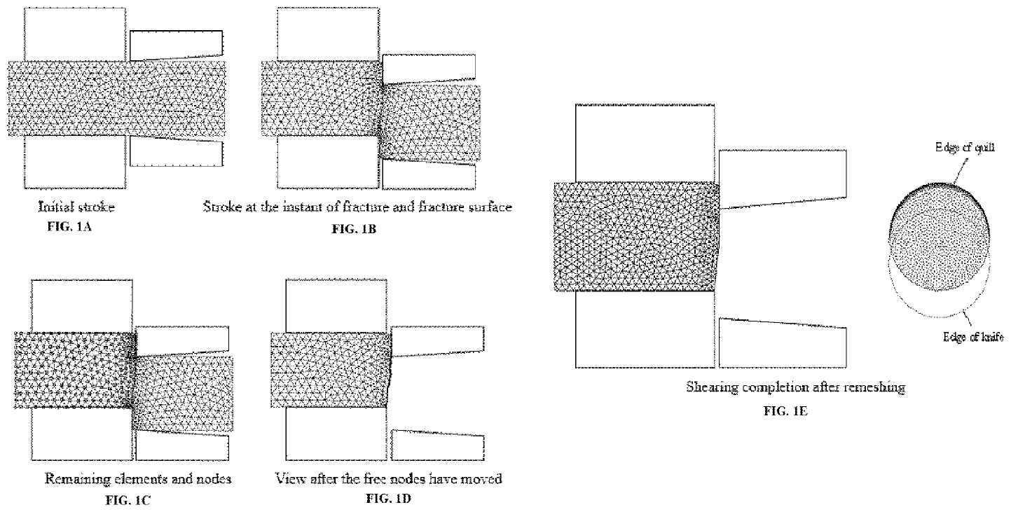

In most cases, 3-D prediction of a sheared material using a damaged element deletion method may not be suitable for finite element analysis of the shearing process for a next metal forming stage. This may result in an inaccurate solution. Therefore, this method may not be suitable for accurate simulation due to extensive volume loss of a material. This problem may be important in terms of the numerical accuracy in some cases, and in the present disclosure, the sheared surface quality control system may be used to obtain a sheared material (finite element model) flawless to the finite element usability or suitability. FIGS. 1A to 1E are views showing a concept with a new system.

At describing the entire shearing process simulation method of the present disclosure, the present disclosure relates to a shearing process simulation method to obtain a numerical preform or billet that may be used for engineering analysis of a consecutive process immediately after shearing, and the shearing process simulation method may include: discretizing a target object into a finite element and a node, i.e., a finite element mesh system for a material (hereinbelow, which will be referred to as a mesh); in a shearing simulation process, determining a shearing point (point where an inside element strength degraded area (which will be referred to as a shearing boundary area) is formed and thus the entire forming load is sharply reduced) by using a shearing point prediction function (element strength degrading algorithm and ductile fracture theory); determining a deleting target area on the basis of the shearing boundary and finding a node that is a deleting target (which will be referred to as a free node) included in the deleting target area; locating the free node in the shearing boundary by applying a constant tensile force to all line segments connected to each other through the free node and extremely reducing an unbalance force or a resistance force exerting onto a material (or in a corresponding method such as node smoothing, etc.), thereby generating a mesh containing the degenerate finite element; assigning a state variable to the free node as a nodal value of a non-deletion node close to each free node, and assigning a state variable to the finite element defined with the free node, and performing re-meshing for the modified mesh containing the degenerate element newly moved, i.e., an artificial mesh before the numerical preform or billet unsuitable for analysis, thereby, with the re-meshing, deleting the degenerate finite element collected in the shearing boundary to generate a mesh that is flawless in terms of analysis of the finite element.

FIGS. 1A to 1E may show the entire process of the shearing process simulation of the present disclosure.

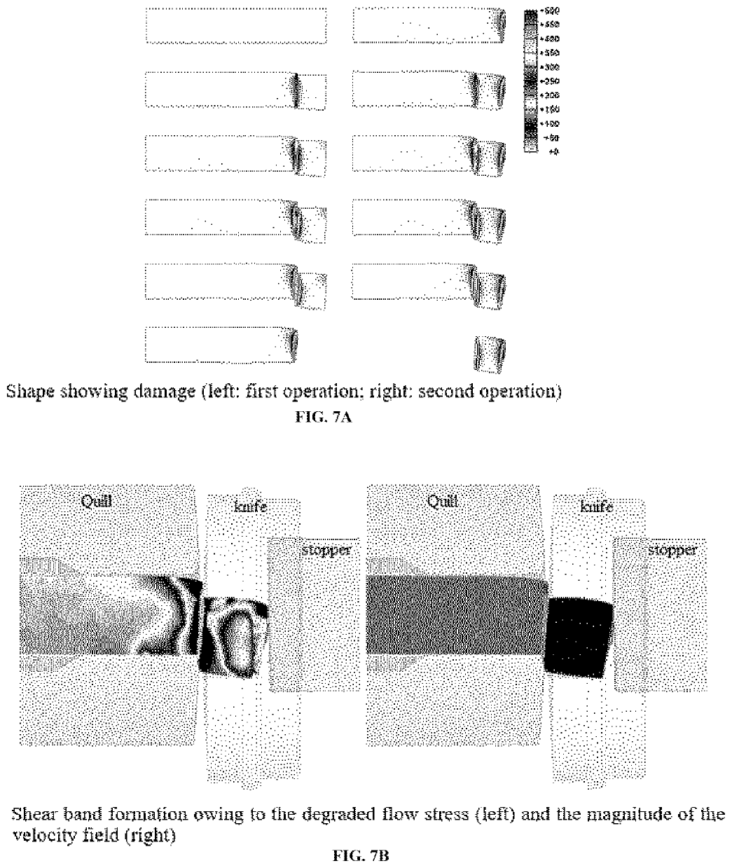

FIG. 1A may be a view showing initial configuration of shearing simulation. FIG. 1B may be a view showing a finite element mesh at the moment of fracture while emphasizing a severely damaged sheared surface (an embodiment thereof may be shown in FIGS. 7A and 7B) where a material is divided into two parts. Some elements remain, while other elements may be deleted. FIG. 1C may show predicted deformation having a shape in which the material is divided into two parts by a sheared surface immediately before applying a separation method. A circled node may be considered to remain after a fracture analysis based on a result of damaging model or experiment or may be selected. When all elements of a specific node are deleted, the node may be excluded by applying an element deletion method. Specifically, the sheared surface may be directly defined by a surface generated after element deletion. However, it may be difficult to apply the method to the actual engineering process due to geometric complexity.

Actually, an element to be deleted may not be deleted during numerical procedure applied to prepare a predicted sheared material. For example, a vanishing node may be arranged at the optimum point by applying a smoothing method. A node averaging method may be applied, and the node averaging method may be configured such that the same tensile force is applied to all edges defined by two vanishing nodes which are called as free nodes. Then, the finite element mesh may become an imaginary mesh similar to a view shown in FIG. 1D. Even when the imaginary mesh is maintained in element connection, the imaginary mesh does not have positive Jacobian. In the new mesh, the nodal value of the free node may be replaced by a nodal value of a node closest thereto. Likewise, an elemental value of an element affected may be estimated in an arithmetic mean of the nodal value. Lastly, as shown in FIG. 1E, after the shearing process simulation, the re-meshing process that is inevitable in any case may be performed. The proposed general and systemic shearing analysis approach may sufficiently reflect macroscopic phenomenon including a predicted shearing shape and strain hardening around a shearing surface which strongly impact on the shearing process, as described above. The numerical preform or billet is flawless in terms of the finite element suitability and usability.

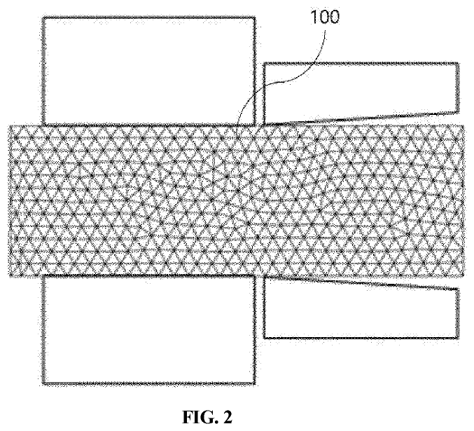

Referring to FIGS. 2 to 6, another embodiment of the present disclosure will be described.

The shearing process simulation method of the present disclosure may include: a first step in which a finite element and a node are generated in a raw material; a second step in which a fracture surface is calculated for a sheared material and a shearing force is applied to the raw material; a third step in which an element of the sheared material is divided into a first group and a second group with the fracture surface as a boundary; a fourth step in which information of a second group element or a second group node included in the second group is averaged to obtain an average value; and a fifth step in which a final fracture surface is generated by reflecting the average value in the fracture surface.

The first step to the fifth step may be computerized by a simulation part of a computer.

The simulation part may recognize the final fracture surface as a boundary surface and may perform a re-meshing process in which an element or a node is regenerated for a remaining part of the sheared material.

The remaining part of the sheared material may be considered as the first group. The simulation part may regenerate a first group element or a first group node of the first group with the final fracture surface as the boundary surface.

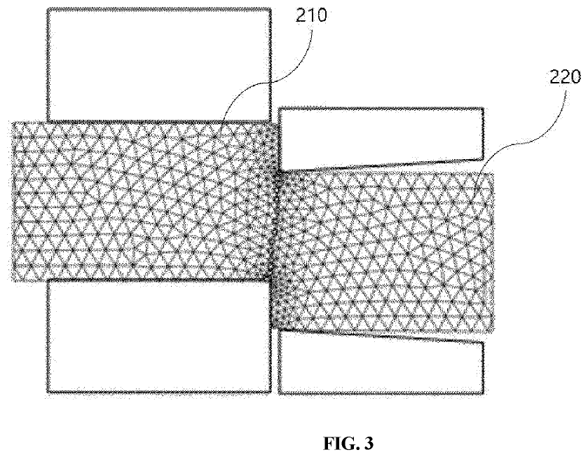

Referring to FIG. 3, the simulation part may recognize a node located in the fracture surface as a fracture surface node and differentiate the node from other nodes. The differentiated fracture surface node may be marked with a circle.

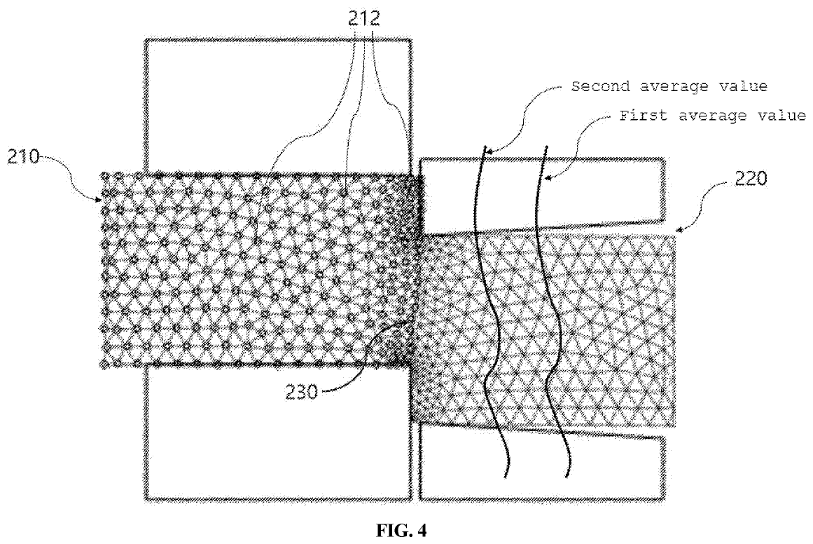

Referring to FIG. 4, the simulation part may recognize the node located in the fracture surface as the fracture surface node and may determine the fracture surface node and the node included in the first group as remaining nodes. The fracture surface node and the remaining nodes are marked with circles.

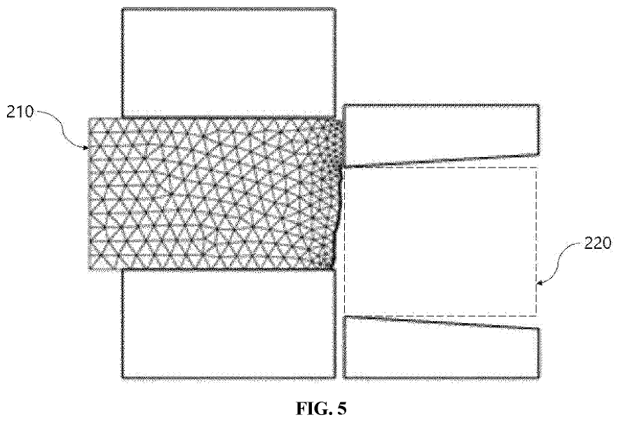

Referring to FIG. 5, conventionally, after the fracture surface is generated, all of a second group element or a second group node may be deleted. When some elements of the sheared material are deleted, sharp discontinuity or deviation of information occurs and thus a simulation error may occur. The fracture surface of FIG. 3 which is generated by the simulation part and a fracture surface in an actual shearing process of the raw material do not match with each other. This is because the fracture surface shown in FIG. 3 is a fracture surface that is simulated by a computer using the finite element method. When the simulated fracture surface is replaced by the actual fracture surface and the second group is deleted, a simulation error may occur owing to out of control.

The present disclosure has a characteristic in which the fracture surface is corrected by smoothing without deleting all information of the second group.

The present disclosure is characterized in that the second group element or the second group node is not deleted before the final fracture surface is generated.

The simulation part may recognize the corrected fracture surface as the boundary surface. The re-meshing corresponding to an element regenerating process may be performed after the boundary surface is generated or during the correcting process of the fracture surface at any time.

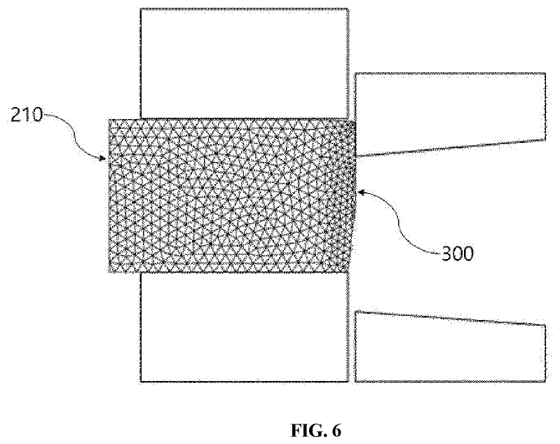

Referring to FIG. 6, information of the second group element or the second group node included in the second group may be reflected in the final fracture surface.

Referring to FIG. 4, the first average value may be generated by averaging information of the second group element or the second group node, and the second average value may be generated by averaging fracture surface information of a node located in the fracture surface and the first average value. This process may be repeated. A Nth average value is generated by averaging the fracture surface information and the N-1th average value, and information of the final fracture surface may be generated on the basis of the Nth average value, and the number N may be a natural number equal to or greater than 3.

Another embodiment of calculating an average value, which is not shown in FIG. 4, will be described as follows.

With the smoothing method, the average value may be calculated by the node averaging method applying the same tensile force to all edges defined by the second group node.

Another embodiment of calculation of the average value not shown in FIG. 4 will be described as follows.

The second group node may be gradually moved to a point close to the fracture surface by the average value. The imaginary mesh may be regenerated whenever moving. This process may be repeated.

A nodal value of the second group node in the imaginary mesh may be replaced by a nodal value of a node closest thereto, and an elemental value of the second group element may be replaced by an arithmetic mean of the nodal value.

When the sheared billet is short, the quality thereof may depend more on the shearing process. A typical example may be a precision cold forging process and may be composed of the shearing process and upsetting process. When knife strokes reach a given value, fracture may occur. The sheared short billet does not have a non-deformed area.

FIGS. 7A and 7B may show plastic deformation and fracture for a predetermined stroke experienced by the shearing process. Furthermore, FIGS. 7A and 7B may emphasize a shearing band completely formed immediately before fracture occurs. The two consecutive shearing processes may be simulated.

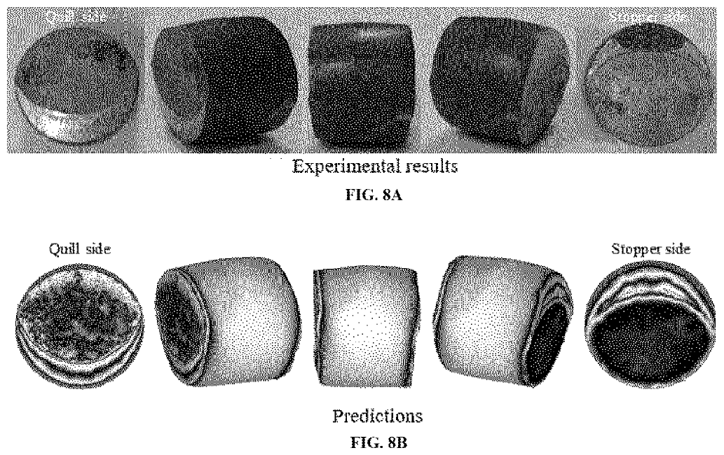

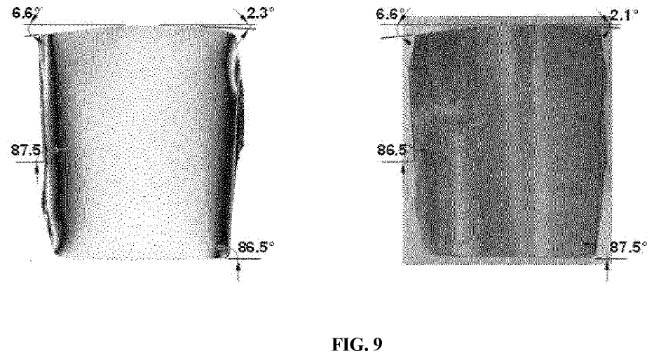

Referring to FIGS. 8A and 8B, the predictions are in close agreement with the experimental results. An upper portion may be bent relatively more than a portion on the opposite side thereto, and the opposite portion may hardly be deformed. As shown in a right view of FIG. 9, inclined angles measured from sheared surfaces at a quill side and a stopper side may be 6.6° and 2.1° compared to lower portions. As shown in a left view of FIG. 9, 6.6° and 2.1° may be in close agreement with 6.6° and 2.3° that are corresponding to predictions.

In a precision metal forming simulation, considering the usability or conformality for the next metal forming step, the description may be sufficient to highlight the importance of shearing analysis.

The present disclosure relates to the new general FEM for shearing, piercing, and blanking, and in the automatic multi-stage metal forming, the usability of a target numerically-sheared material or the finite element suitability may be emphasized. The present disclosure may concentrate the billet shearing based on the fracture theory. Through the simulation and the experimental results, the present disclosure is strong, and specifically, is strong to the precision shearing, blanking, piercing, or trimming process, and the process may be performed under a high compressive stress state in order to adjust burr formation in which minimum scrap is formed.

The proposed methodology may be based on element strength degradation method and be unified with a sheared surface quality control system. The element strength degradation can avoid potentially difficult numerical problems due to excessive re-meshing known in the element deletion and node separation method, and specifically, also avoid a self-contact problem on a sheared surface of the compressive stress state. Therefore, strength degraded element does not worsen convergence solution, and thus universality of the shearing process simulation can be ensured. A low bound having a level of 10% of a normal flow stress, which is assigned to severely damaged elements, may be suitable for convergence of a stable solution with negligible effect on the solution.

The sheared surface quality control system that may be essential to ensure the generality and the usability of the 3-D shearing simulation may give a proper finite element mesh system for the sheared material. This is because a geometrically damaged surface may be modeled into a balloon under a tension having only a local impact on the irregular surface of the material after deleting the severely damaged elements. Since the sheared surface quality control system is configured to separate a material with a single step, so that when selection of an element to be deleted depends on the theory of ductile fracture, the selection of element can be only performed when the element strength degradation model is combined thereto.

During the automatic multi-stage cold forging process, when considering a shearing method based on element strength degradation and a 3-D round-bar shearing process, the usability and validity of the new methodology for 3-D application can be demonstrated by an axisymmetric deep piercing process.

For example, the normalized Cockcroft-Latham damage model and the modified Freudenthal damage model may be used for the validity and the usability. The predictions and the experimental results may be compared to each other to demonstrate the usability of the sheared material as a numerical preform or billet, and no additional numerical processing is required and the two results agree very well with each other.

Most engineering analyses of the shearing process may begin with an idealized billet shape having a uniform initial state variables, and may require a proper numerical method. The approach proposed in the present disclosure may be directly applied to the 3-D shearing process for precision simulation. The material may be accurately sheared or blanked under the compressive stress state.

1. A shearing process simulation method comprising:
a first step in which a finite element and a node are generated in a raw material;
a second step in which a fracture surface is calculated for a sheared material with a shearing force applied to the raw material;
a third step in which an element of the sheared material is divided into a first group and a second group with the fracture surface as a boundary;
a fourth step in which an average value is obtained by averaging information of a second group element or a second group node included in the second group; and
a fifth step in which a final fracture surface is generated by reflecting the average value in the fracture surface,
wherein the final fracture surface is recognized as a boundary surface.

2. The shearing process simulation method of claim 1, wherein a first group element or a first group node of the first group is regenerated with the final fracture surface as the boundary surface.

3. The shearing process simulation method of claim 1, wherein a node located in the fracture surface is recognized as a fracture surface node and is divided from other nodes.

4. The shearing process simulation method of claim 1, wherein a node located in the fracture surface is recognized as a fracture surface node and the fracture surface node and a node included in the first group are determined as remaining nodes.

5. The shearing process simulation method of claim 1, wherein information of the second group element or the second group node included in the second group is reflected in the final fracture surface.

6. The shearing process simulation method of claim 1, wherein a first average value is generated by averaging information of the second group element or the second group node,
a second average value is generated by averaging fracture surface information of a node located in the fracture surface and the first average value,
an Nth average value is generated by averaging the fracture surface information and an N-1th average value,
information of the final fracture surface is generated based on the Nth average value, and
the number N is a natural number greater than or equal to 3.

7. The shearing process simulation method of claim 1, wherein the average value is calculated using a node averaging method in which an equal tensile force is applied to all edges defined by the second group node.

8. The shearing process simulation method of claim 1, wherein before the final fracture surface is generated, the second group element or the second group node is not deleted.

9. The shearing process simulation method of claim 1, wherein the second group node is moved to a point close to the fracture surface the average value,
an imaginary mesh is regenerated, and
a nodal value of the second group node in the imaginary mesh is replaced by a nodal value of a node closest thereto, and an elemental value of the second group element is replaced by an arithmetic mean of the nodal value.

10. A shearing process simulation method comprising:
a first step in which by a finite element mesh system for a material, the material is divided into a finite element and a node;
a second step in which in a shearing simulation process, element strength degrading algorithm and a shearing point prediction function of ductile fracture theory are used to determine a shearing point, or a shearing boundary area that is an element strength degraded area in a raw material is generated so that the entire forming load is sharply degraded;
a third step in which a deleting target area is determined based on the shearing boundary, and a free node that is a deleting target included in the deleting target area is generated;
a fourth step in which a constant tensile force is applied to all line segments connected to each other by the free node, and an unbalance or resistance force of the material is reduced by placing the free node at the appropriate position or a node smoothing method is applied, the free node is located in a shearing boundary, and a mesh containing a degenerate finite element is regenerated;
a fifth step in which a state variable value is assigned to the free node as a nodal value of a non-deletion node close to each free node, and the state variable value is assigned to the finite element defined by the free node; and
a sixth step in which a numerical preform or billet sheared in the shearing simulation is formed, and a re-meshing is performed with respect to a mesh containing the degenerate finite element of the numerical preform or billet and thus a strength degraded finite element collected in the shearing boundary is deleted, and a flawless mesh is generated in terms of finite element analysis,
wherein through the first step to the sixth step, a numerical preform or billet that is able to be used for engineering analysis of a consecutive metal forming process immediately after the shearing process is obtained.
